(19) United States
(12) Patent Application Publication
(10) Pub. No.: US 2026/0237495 A1
(43) Pub. Date: Aug. 13, 2026

(54) SYSTEM AND METHOD FOR ASSISTING A CONSUMER WITH SERVICE ENROLLMENT VIA AN AI-ASSISTED REASONING ENGINE

(71) Applicant: Fernando Sotelo, North Richland Hills, TX (US)

(72) Inventor: Fernando Sotelo, North Richland Hills, TX (US)

(21) Appl. No.: 19/052,204

(22) Filed: Feb. 12, 2025

(51) Int. Cl. G16H 40/20; G06Q 40/08; G16H 15/00

(52) U.S. Cl. CPC G16H 40/20; G06Q 40/08; G16H 15/00

(57) ABSTRACT

A method for simplifying and accelerating the U.S. individual healthcare enrollment process involves using a conversational artificial intelligence (AI) agent. The AI agent interacts with a consumer via a web app or mobile platform, asking a series of tailored questions related to healthcare coverage, eligibility, and enrollment. Based on client responses, the AI agent helps navigate the healthcare marketplace, provides real-time information, and assists in selecting the most suitable insurance policy. The AI also stores user data, explains coverage documents in simple terms, and finalizes the selected policy without requiring a live agent unless complex issues arise. This system aims to reduce enrollment time, enhance client understanding, and mitigate fraud risks. The platform is an Enhanced Direct Enrollment (EDE) solution, approved by CMS and Healthcare.gov, ensuring secure and efficient policy submission.

### TECHNICAL FIELD

The embodiments generally relate to the technical field of automated service enrollment and customer service, and in particular, the use of artificial intelligence-based conversational agents to assist U.S. consumers with navigating, for example, the individual healthcare marketplace, answering related questions, guiding clients through the enrollment process, and finalizing policy selections.

### BACKGROUND

Traditional methods for individual healthcare enrollment in the United States involve lengthy and often complex processes that require consumers to interact with live agents or navigate through extensive paperwork. Typically, consumers must visit healthcare.gov or other insurance platforms, answer a series of questions, and wait for a human representative to assist in finalizing their coverage. This process can be confusing, especially for those unfamiliar with health insurance, resulting in a slow, cumbersome, and frustrating experience. Furthermore, many individuals have questions about their coverage, eligibility, and the laws surrounding the Affordable Care Act, requiring additional time and effort to resolve.

Despite the availability of online resources and digital forms, the process of enrolling in individual healthcare coverage remains inefficient. Current methods still heavily rely on phone calls or in-person consultations, which can create delays, increase the likelihood of errors, and often lead to consumer dissatisfaction. In addition, the complexity of health insurance terminology and the numerous plan options available can overwhelm many consumers, leaving them with an incomplete understanding of their coverage or potentially making uninformed decisions.

Existing solutions have not leveraged conversational artificial intelligence (AI) to streamline this process. While some platforms offer online tools or automated prompts, these systems often lack the interactive capabilities to guide clients effectively or answer complex questions in real time. These tools do not provide the same level of personalized support as a human agent, and as a result, the process remains inefficient and prone to error. Furthermore, current systems are not optimized for security and fraud mitigation, which poses significant risks to both consumers and healthcare providers.

The need for a more efficient, secure, and client-friendly solution has become increasingly apparent. A system that allows consumers to navigate the healthcare enrollment process through a conversational AI agent could not only reduce the time and effort required to finalize policies but also improve the accuracy of information provided, minimize errors, and help address issues related to fraud and security. Such a system would revolutionize the healthcare enrollment experience, making it accessible, clear, and efficient for all demographics across the United States.

### SUMMARY

This summary is provided to introduce a variety of concepts in a simplified form that are further disclosed in the detailed description of the embodiments. This summary is not intended to identify key or essential inventive concepts of the claimed subject matter, nor is it intended to determine the scope of the claimed subject matter.

In some aspects, the system includes at least one computing device in operable communication with a network and an application server that hosts a conversational service AI agent. The application program may be configured to engage with a client through a series of interactive prompts, receiving responses to questions related to service marketplace coverage, eligibility, and the enrollment process. The AI agent is capable of answering complex questions, guiding clients through the enrollment process via questions and answers, and helping them select the most suitable policy based on their individual circumstances.

In one aspect, the AI agent is capable of conducting an interview-style dialogue with the client, asking a series of questions tailored to the consumer's needs and preferences. These prompts are dynamically generated and refined by the AI, utilizing natural language processing (NLP) to interpret and adjust based on the user's responses. The AI agent can provide accurate, real-time information about healthcare plans, the Affordable Care Act, and coverage options without requiring human intervention unless a complex issue arises.

In another aspect, the AI agent uses a large language model (LLM) to intelligently analyze user input and adjust its questions accordingly to ensure the user's responses are relevant and accurate. This iterative process allows the system to refine its understanding of the user's needs, ultimately leading to the selection of the best possible service plan. Once the enrollment process is completed, the AI agent can provide a summary of the selected policy and offer further assistance with understanding the details of the coverage.

In one embodiment, the system is designed to ensure a secure and efficient enrollment experience by adhering to government guidelines and being an Enhanced Direct Enrollment (EDE) platform approved by the Centers for Medicare & Medicaid Services (CMS) and Healthcare.gov. This approval allows the system to securely submit enrollment information and finalize policies without the need for live agent interaction, reducing the time and potential for errors in the process.

Other illustrative variations within the scope of the invention will become apparent from the detailed description provided hereinafter. The detailed description and enumerated variations, while disclosing optional variations, are intended for purposes of illustration only and are not intended to limit the scope of the invention.

### BRIEF DESCRIPTION OF THE DRAWINGS

A more complete understanding of the embodiments, and the attendant advantages and features thereof, will be more readily understood by references to the following detailed description when considered in conjunction with the accompanying drawings wherein:

FIG. 1 illustrates a system architecture diagram, according to some embodiments;

FIG. 2 illustrates an application program and modules in communication with the computing system, according to some embodiments;

FIG. 3 illustrates a flowchart illustrating a computer-implemented user interview process, according to some embodiments;

FIG. 4 illustrates a flowchart illustrating a computer-implemented AI report generation and refinement process, according to some embodiments; and

FIG. 5 illustrates a flowchart illustrating a computer-implemented data security and compliance process, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing exemplary embodiments in detail, it is noted that the embodiments reside primarily in combinations of components related to devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

The invention relates to a system and method that utilizes AI to assist U.S. consumers with service enrollment in a straightforward, efficient, and secure manner. As used herein, “service” and “healthcare” are used interchangeably to refer to the same grouping of services: healthcare enrollment, life insurance enrollment, supplement services enrollment, or general enrollment in various services. This AI-driven system employs conversational agents to guide users through the healthcare marketplace enrollment process, helping them select appropriate health insurance policies without the necessity of direct interaction with human agents, unless required for more complex issues.

The invention comprises a combination of device components, including computing devices, user interfaces, and AI models. The accompanying diagrams use conventional symbols and system schematics to illustrate these components, focusing on the essential details necessary for understanding the invention while avoiding unnecessary complexity.

The system features an AI-assisted reasoning engine that leverages large language models (LLMs) for various tasks, such as interpreting user responses, generating relevant follow-up prompts, and dynamically refining the interview process. This engine powers a user-friendly platform accessible via a smartphone app or web interface, where users can engage with the AI agent to provide personal and eligibility-related information. The AI agent can pose follow-up questions, guide users through the marketplace, and assist them in selecting the most suitable insurance plan based on their specific needs. Once the interview is complete, the system automatically generates a summary report detailing the consumer's selected policy. This report is then forwarded to the insurance provider or healthcare organization for processing. The system is designed with robust security and privacy features to comply with regulations such as the Health Insurance Portability and Accountability Act (HIPAA) and the General Data Protection Regulation (GDPR). It ensures end-to-end encryption of personal and policy data, secure storage, and access control measures. Furthermore, the system promotes collaboration among users, insurance providers, and healthcare professionals by enabling secure data sharing among authorized parties, thereby streamlining the enrollment and policy management process.

Implementations of the invention fall under the technical field of AI-based healthcare systems, including interview automation, report generation, and data analysis. The system performs tasks such as delivering interview prompts, collecting user responses, and generating a policy selection analysis. These functions utilize computer technology, particularly AI models and natural language processing (NLP) techniques, to refine the interview process and address issues such as incomplete or inconsistent user data. Unlike traditional forms or static questionnaires found in typical electronic health record (EHR) systems, this system adapts to the user's responses and provides dynamic, real-time assistance. The platform can communicate with users in multiple languages, making it accessible to diverse populations and facilitating inclusive healthcare delivery.

The steps involved in this method and system are inherently computer-based, as they rely on advanced AI models to dynamically analyze responses, generate targeted interview questions, and produce comprehensive reports to assist consumers in health insurance selection. These processes cannot be done manually or mentally, as they depend on the AI model's ability to process large volumes of data in real time while ensuring consistency and accuracy. The invention represents a significant improvement over traditional manual or static systems by offering an AI-driven, user-centric platform that reduces enrollment time, increases accuracy, and minimizes the need for human intervention unless necessary for complex cases.

The present invention makes use of advanced AI models, such as an LLM,, which are deployed on computing devices. These devices execute the models' algorithms and mathematical functions, enabling the system to process large datasets in real-time. As the AI model is trained on healthcare-specific datasets, it continually enhances its understanding of user input and refines its responses and recommendations. The system's capability to analyze and process data on a large scale distinguishes it from traditional methods, which would struggle to manage the volume and complexity of data required for automating healthcare enrollment and policy selection. Employing AI for this purpose provides a clear technical improvement over previous healthcare enrollment methods, ensuring faster, more accurate, and more efficient outcomes for consumers.

Additionally, the system may include an AI-assisted reasoning engine, designed to conduct client interviews based on responses to interview prompts. A client-centric platform allows clients to interact with the system, typically personalizing the experience to better meet their service needs.

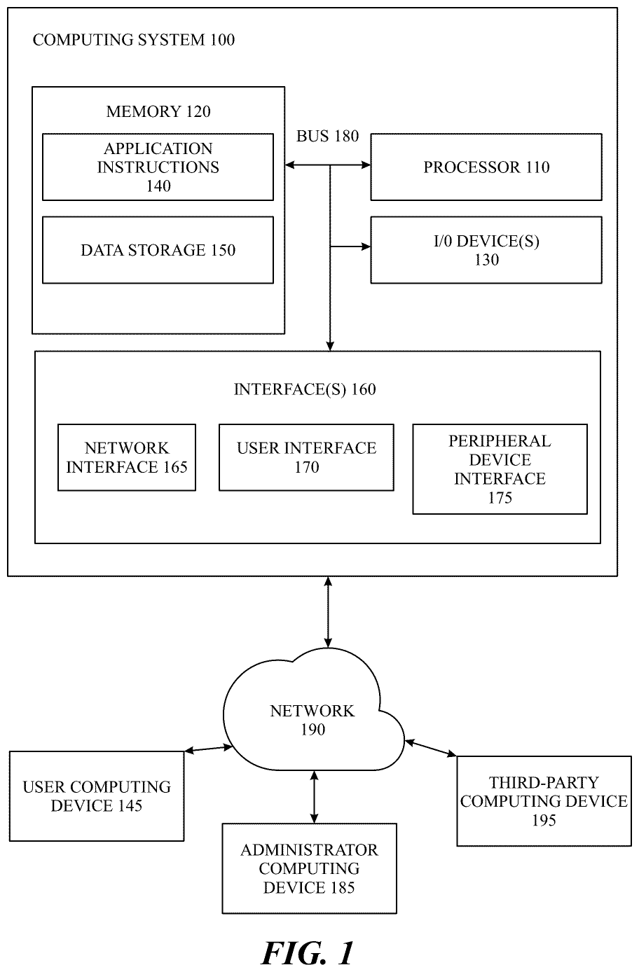

FIG. 1 illustrates an example of a computer system 100 that may be utilized to execute various procedures, including the processes described herein. The computer system 100 comprises a standalone computer or mobile computing device, a mainframe computer system, a workstation, a network computer, a desktop computer, a laptop, or the like. The computer system 100 can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

In some embodiments, the computer system 100 includes one or more processors 110 coupled to a memory 120 through a system bus 180 that couples various system components, such as an input/output (I/O) devices 130, to the processors 110. The bus 180 may be any of several types of bus structures including a memory bus or memory controller, a peripheral bus, and a local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

In some embodiments, the computer system 100 includes one or more input/output (I/O) devices 130, such as video device(s) (e.g., a camera), audio device(s), and display(s) are in operable communication with the computer system 100. In some embodiments, similar I/O devices 130 may be separate from the computer system 100 and may interact with one or more nodes of the computer system 100 through a wired or wireless connection, such as over a network interface.

Processors 110 suitable for the execution of computer readable program instructions include both general and special purpose microprocessors and any one or more processors of any digital computing device. For example, each processor 110 may be a single processing unit or a number of processing units and may include single or multiple computing units or multiple processing cores. The processor(s) 110 can be implemented as one or more microprocessors, microcomputers, microcontrollers, digital signal processors, central processing units, state machines, logic circuitries, and/or any devices that manipulate signals based on operational instructions. For example, the processor(s) 110 may be one or more hardware processors and/or logic circuits of any suitable type specifically programmed or configured to execute the algorithms and processes described herein. The processor(s) 110 can be configured to fetch and execute computer readable program instructions stored in the computer-readable media, which can program the processor(s) 110 to perform the functions described herein.

In this disclosure, the term “processor” can refer to substantially any computing processing unit or device, including single-core processors, single-processors with software multithreading execution capability, multi-core processors, multi-core processors with software multithreading execution capability, multi-core processors with hardware multithread technology, parallel platforms, and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures, such as molecular and quantum-dot based transistors, switches, and gates, to optimize space usage or enhance performance of user equipment. A processor can also be implemented as a combination of computing processing units.

In some embodiments, the memory 120 includes computer-readable application instructions 140, configured to implement certain embodiments described herein, and a database 150, comprising various data accessible by the application instructions 140. In some embodiments, the application instructions 140 include software elements corresponding to one or more of the various embodiments described herein. For example, application instructions 140 may be implemented in various embodiments using any desired programming language, scripting language, or combination of programming and/or scripting languages (e.g., Android, C, C++, C #, JAVA, JAVASCRIPT, PERL, etc.).

In this disclosure, terms “store,” “storage,” “data store,” data storage,” “database,” and substantially any other information storage component relevant to operation and functionality of a component are utilized to refer to “memory components,” which are entities embodied in a “memory,” or components comprising a memory. Those skilled in the art would appreciate that the memory and/or memory components described herein can be volatile memory, nonvolatile memory, or both volatile and nonvolatile memory. Nonvolatile memory can include, for example, read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include, for example, RAM, which can act as external cache memory. The memory and/or memory components of the systems or computer-implemented methods can include the foregoing or other suitable types of memory.

Generally, a computing device will also include or be operatively coupled to receive data from or transfer data to, or both, one or more mass data storage devices; however, a computing device need not have such devices. The computer readable storage medium (or media) can be a tangible device that can retain and store instructions for use by an instruction execution device. The computer readable storage medium can be, for example, an electronic storage device, a magnetic storage device, an optical storage device, an electromagnetic storage device, a semiconductor storage device, or any suitable combination of the foregoing. A non-exhaustive list of more specific examples of the computer readable storage medium can include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. In this disclosure, a computer readable storage medium is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, the steps and actions of the application instructions 140 described herein are embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions 140 for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions 140 can execute entirely on the user's computer, partly on the user's computer, as a stand-alone software package, partly on the user's computer and partly on a remote computer, or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

In some embodiments, the application instructions 140 can be downloaded to a computing/processing device from a computer readable storage medium, or to an external computer or external storage device via a network 190. A network adapter card or network interface in each computing/processing device receives computer readable program instructions from the network and forwards the computer readable application instructions 140 for storage in a computer readable storage medium within the respective computing/processing device.

In some embodiments, the computer system 100 includes one or more interfaces 160 that allow the computer system 100 to interact with other systems, devices, or computing environments. In some embodiments, the computer system 100 comprises a network interface 165 to communicate with a network 190. In some embodiments, the network interface 165 is configured to allow data to be exchanged between the computer system 100 and other devices attached to the network 190, such as other computer systems, or between nodes of the computer system 100. In various embodiments, the network interface 165 may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol. Other interfaces include the user interface 170 and the peripheral device interface 175.

In some embodiments, the network 190 corresponds to a local area network (LAN), wide area network (WAN), the Internet, a direct peer-to-peer network (e.g., device to device Wi-Fi, Bluetooth, etc.), and/or an indirect peer-to-peer network (e.g., devices communicating through a server, router, or other network device). The network 190 can comprise copper transmission cables, optical transmission fibers, wireless transmission, routers, firewalls, switches, gateway computers and/or edge servers. The network 190 can represent a single network or multiple networks. In some embodiments, the network 190 used by the various devices of the computer system 100 is selected based on the proximity of the devices to one another or some other factor. For example, when a first user device and second user device are near each other (e.g., within a threshold distance, within direct communication range, etc.), the first user device may exchange data using a direct peer-to-peer network. But when the first user device and the second user device are not near each other, the first user device and the second user device may exchange data using a peer-to-peer network (e.g., the Internet). The Internet refers to the specific collection of networks and routers communicating using an Internet Protocol (“IP”) including higher level protocols, such as Transmission Control Protocol/Internet Protocol (“TCP/IP”) or the Uniform Datagram Packet/Internet Protocol (“UDP/IP”).

Any connection between the components of the system may be associated with a computer-readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. As used herein, the terms “disk” and “disc” include compact disc (CD), laser disc, optical disc, digital versatile disc (DVD), floppy disk, and Blu-ray disc; in which “disks” usually reproduce data magnetically, and “discs” usually reproduce data optically with lasers. Combinations of the above should also be included within the scope of computer-readable media. In some embodiments, the computer-readable media includes volatile and nonvolatile memory and/or removable and non-removable media implemented in any type of technology for storage of information, such as computer-readable instructions, data structures, program modules, or other data. Such computer-readable media may include RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. Depending on the configuration of the computing device, the computer-readable media may be a type of computer-readable storage media and/or a tangible non-transitory media to the extent that when mentioned, non-transitory computer-readable media exclude media such as energy, carrier signals, electromagnetic waves, and signals per se.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In some embodiments, the system can also be implemented in cloud computing environments. In this context, “cloud computing” refers to a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

As used herein, the term “add-on” (or “plug-in”) refers to computing instructions configured to extend the functionality of a computer program, where the add-on is developed specifically for the computer program. The term “add-on data” refers to data included with, generated by, or organized by an add-on. Computer programs can include computing instructions, or an application programming interface (API) configured for communication between the computer program and an add-on. For example, a computer program can be configured to look in a specific directory for add-ons developed for the specific computer program. To add an add-on to a computer program, for example, a user can download the add-on from a website and install the add-on in an appropriate directory on the user's computer.

In some embodiments, the computer system 100 may include a user computing device 145, an administrator computing device 185 and a third-party computing device 195 each in communication via the network 190. The user computing device 145 may be utilized by a user to interact with the various functionalities of the system. The administrator computing device 185 is utilized by an administrative user to moderate content and to perform other administrative functions. The third-party computing device 195 may be utilized by third parties to receive communications from the user computing device, transmit communications to the user via the network, and otherwise interact with the various functionalities of the system.

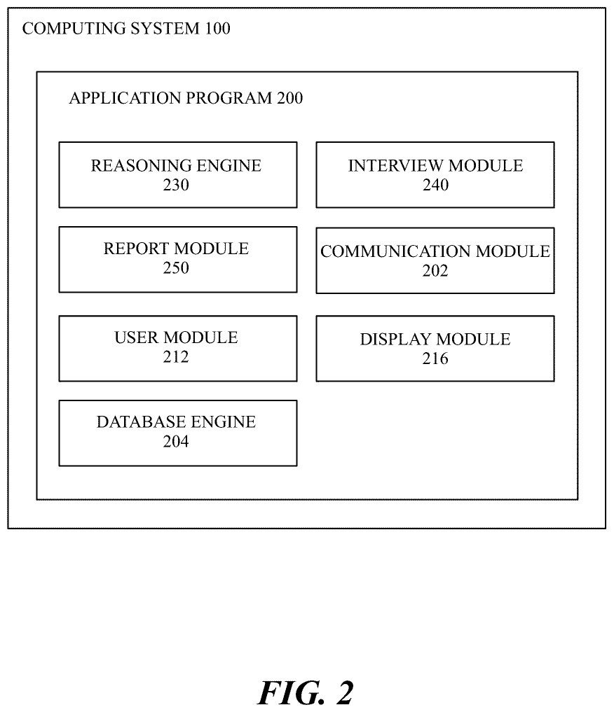

FIG. 2 illustrates an example computer architecture for the application program 200 operated via the computing system 100. The computer system 100 comprises several modules and engines configured to execute the functionalities of the application program 200, and a database engine 204 configured to facilitate how data is stored and managed in one or more databases. In particular, FIG. 2 is a block diagram showing the modules and engines needed to perform specific tasks within the application program 200.

Referring to FIG. 2, the computing system 100 operating the application program 200 comprises one or more modules having the necessary routines and data structures for performing specific tasks, and one or more engines configured to determine how the platform manages and manipulates data. In some embodiments, the application program 200 comprises one or more of an AI-based reasoning engine 230, an interview module 240, a report module 250, a communication module 202, a database engine 204, a user module 212, and a display module 216.

In some embodiments, the AI-based reasoning engine 230 is configured to supplement the performance of a conversational agent or avatar employed by the interview module 240 by integrating knowledge representation, logical inference, and probabilistic modeling to process health insurance marketplace data efficiently. In embodiments, the AI agent is configurable based on client preferences, including, but not limited to, language preferences, AI agent avatar appearance preference, text-to-speech audio preferences, etc. In embodiments, the AI agent includes an avatar that may be configured to visualize the AI agent, and which may be turned on or off at the preference of the client. The AI-based reasoning engine 230 is configured to structure data using knowledge graphs, ontologies, and rule-based systems, enabling logical inference through deductive and inductive reasoning. The AI-based reasoning engine 230 dynamically generates SQL/API queries, leveraging NLU models to extract client intent and key entities from conversational information. For decision-making, the AI-based reasoning engine 230 applies constraint optimization (e.g., minimizing out-of-pocket costs) and probabilistic inference (e.g., Bayesian models for plan recommendations). The AI-based reasoning engine 230 may use multi-turn dialog management ensures context retention and reinforcement learning to refine responses based on client feedback. The AI-based reasoning engine 230 may include compliance enforcement mechanisms to validate responses against regulatory frameworks (e.g., HIPAA and GDPR), mitigating misinformation risks.

In some embodiments, the interview module 240 is configured to employ one or more AI-drive conversational agents, in cooperation with the AI-based reasoning engine 230. The interview module 240 is configured to provide personalized recommendations, simplifying complex terminology, and automating plan comparisons. The interview module 240 is configured to use NLP to break down premiums, deductibles, and out-of-pocket costs while leveraging machine learning to match clients with optimal plans. The interview module 240 may streamline enrollment by verifying eligibility, assisting with document uploads, and sending deadline reminders. The interview module 240 may provide real-time responses to subsidy, renewal, and compliance queries while detecting fraud through anomaly detection models. Post-enrollment, the interview module 240 may facilitate provider searches, claims filing, and coverage adjustments. Multilingual support and data analytics improve accessibility and inform policy refinements, optimizing the client experience.

In this way, the AI-based reasoning engine 230 and the interview module 240 are configured to communicate a first interview prompt to a client device; receive a first response to the first interview prompt; dynamically generating, via an AI-assisted reasoning engine fine-tuned on healthcare-specific data, a second interview prompt based on semantic analysis of the first response, wherein the AI-assisted reasoning engine detects incomplete or inconsistent data and generates targeted follow-up prompts; and guide the client through the healthcare marketplace, answering enrollment-related questions, and assisting in selecting a healthcare policy based on the first response and the second response. In embodiments, the AI-based reasoning engine 230 works in cooperation with the interview module 240 to process client requests or questions regarding service or insurance information, such as policies, explanations of benefits, or summaries of benefits, via NLP. In this way, the interview module 240 is configured to provide policy-specific responses to client inquiries.

In some embodiments, the report module 250 is configured to generate an analysis and enrollment report based on the first and second responses in cooperation with the AI-based reasoning engine 230 by performing NLP of the interview questions and responses communicated via the conversational agent employed by the interview module 240.

In some embodiments, the communication module 202 is configured for receiving, processing, and transmitting a client command and/or one or more data streams. In such embodiments, the communication module 202 performs communication functions between various devices, including the client computing device 145 of FIG. 1, the administrator computing device 185 of FIG. 1, and a third-party computing device 195 of FIG. 1. In some embodiments, the communication module 202 is configured to allow one or more clients of the system, including a third-party, to communicate with one another. In some embodiments, the communications module 202 is configured to maintain one or more communication sessions with one or more servers, the administrative computing device 185 of FIG. 1, and/or one or more third-party computing device(s) 195 of FIG. 1. In some embodiments, the communication module 202 may allow clients and administrators to communicate with one another.

In some embodiments, a database engine 204 is configured to facilitate the storage, management, and retrieval of data to and from one or more storage mediums, such as the one or more internal databases described herein. In some embodiments, the database engine 204 is coupled to an external storage system. In some embodiments, the database engine 204 is configured to apply changes to one or more databases. In some embodiments, the database engine 204 comprises a search engine component for searching through thousands of data sources stored in different locations.

The user module 212 may store client preferences including the client account information, historical usage data, client personal information, and the like. The user module 212 may facilitate the creation of client's profiles for clients, administrators, and others. Additionally, the user module 212 may utilize two-factor authentication (2FA) to ensure user or client identity validation, including biometric validation, prior to making any changes to client policies, records, etc.

In some embodiments, the display module 216 is configured to display one or more graphic user interfaces, including, e.g., one or more user interfaces. In some embodiments, the display module 216 is configured to temporarily generate and display various pieces of information in response to one or more commands or operations. The various pieces of information or data generated and displayed may be transiently generated and displayed, and the displayed content in the display module 216 may be refreshed and replaced with different content upon the receipt of different commands or operations in some embodiments. In such embodiments, the various pieces of information generated and displayed in a display module 216 may not be persistently stored. The display module 216 displays information, notifications, and alerts to the user device which can be viewed and acknowledged by the client.

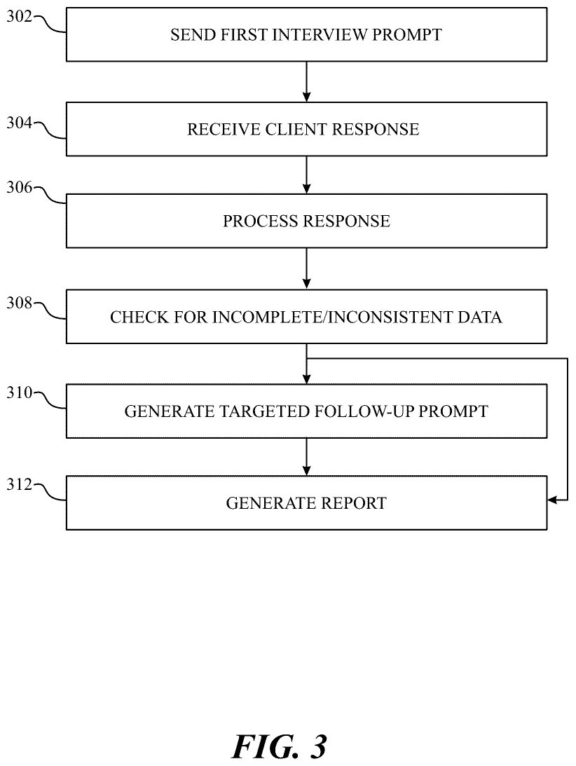

FIG. 3 illustrates a flowchart illustrating a computer-implemented client interview process demonstrating the process of a client providing their medical history via an AI-assisted interview. A client may initiate the interview process and, in step 302, the system sends a prompt to the client's device. In step 304, the client responds to the interview prompt and the system receives the client's response. In step 306, the system analyzes the client's response via the AI-assisted reasoning engine. In step 308, the system analyzes the interview prompts and corresponding client responses to identify incomplete or inconsistent data. If incomplete or inconsistent data is identified, the system proceeds to step 310 and generates follow-up questions to which the client must respond to clarify incomplete or inconsistent data. If no incomplete or inconsistent data is identified, the system proceeds to step 312 and generates a summary report based on responses.

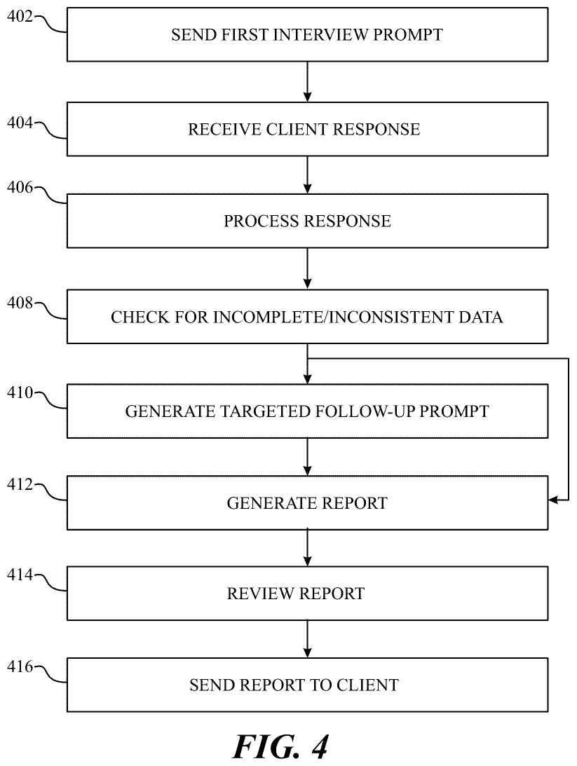

FIG. 4 illustrates a flowchart illustrating a computer-implemented AI report generation and refinement process demonstrating how the AI-assisted reasoning engine generates and refines medical reports based on client inputs. In step 402, the system sends a prompt to the client's device. In step 404, the system receives initial responses from client during the conversational interview. In step 406, the AI-assisted reasoning engine processes input using NLP. In step 408, the system analyzes the interview prompts and corresponding client responses to identify incomplete or inconsistent data. If incomplete or inconsistent data is identified, the system proceeds to step 410 and generates follow-up questions to which the client must respond to clarify incomplete or inconsistent data. If no incomplete or inconsistent data is identified, the system proceeds to step 412 and generates a summary report based on responses. In step 414, the system reviews the generated report for accuracy. In step 416, the system sends the generated report to the client.

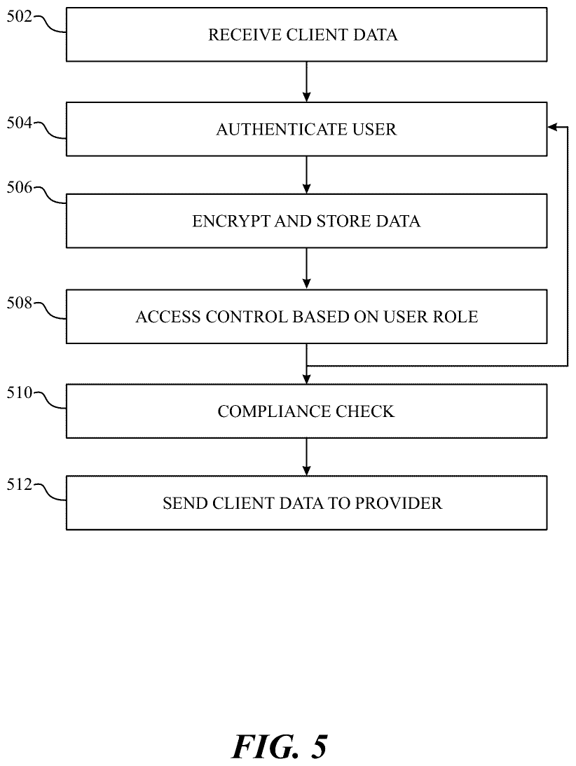

FIG. 5 illustrates a flowchart illustrating a computer-implemented data security and compliance process demonstrating how the system ensures security and compliance with data protection regulations like HIPAA and GDPR when a client, such as a healthcare provider, attempts to access client data. In step 502, the system receives client data, such as via the aforementioned interview process. In step 504, the system uses secure authentication, such as multi-factor authentication, to ensure only authorized clients have access to stored client data. In step 506, the system encrypts and stores client data using AES-256 encryption or similar encryption. In step 508, the system determines if a client attempting to access client data has proper access based on their role. If not, the system returns to step 504 to re-authenticate the client. If yes, the system proceeds to step 510 and performs a compliance check to confirm that all steps taken meet HIPAA/GDPR standards for privacy and security. In step 512, the system allows authorized clients to access encrypted client data.

In this disclosure, the various embodiments are described with reference to the flowchart illustrations and/or block diagrams of methods, apparatus (systems), and computer program products. Those skilled in the art would understand that each block of the flowchart illustrations and/or block diagrams, and combinations of blocks in the flowchart illustrations and/or block diagrams, can be implemented by computer readable program instructions. The computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer readable program instructions can be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can be loaded onto a computer, other programmable data processing apparatus, or other device to cause a series of operational acts to be performed on the computer, other programmable apparatus, or other device to produce a computer implemented process, such that the instructions that execute on the computer, other programmable apparatus, or other device implement the functions or acts specified in the flowchart and/or block diagram block or blocks.

In this disclosure, the block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carry out combinations of special purpose hardware and computer instructions.

In this disclosure, the subject matter has been described in the general context of computer-executable instructions of a computer program product running on a computer or computers, and those skilled in the art would recognize that this disclosure can be implemented in combination with other program modules. Generally, program modules include routines, programs, components, data structures, etc. that perform particular tasks and/or implement particular abstract data types. Those skilled in the art would appreciate that the computer-implemented methods disclosed herein can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be located in both local and remote memory storage devices.

In this disclosure, the terms “component,” “system,” “platform,” “interface,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

The phrase “application” as is used herein means software other than the operating system, such as Word processors, database managers, Internet browsers and the like. Each application generally has its own user interface, which allows a user to interact with a particular program. The user interface for most operating systems and applications is a graphical user interface (GUI), which uses graphical screen elements, such as windows (which are used to separate the screen into distinct work areas), icons (which are small images that represent computer resources, such as files), pull-down menus (which give a client a list of options), scroll bars (which allow a client to move up and down a window) and buttons (which can be “pushed” with a click of a mouse). A wide variety of applications is known to those in the art.

The phrases “Application Program Interface” and API as are used herein mean a set of commands, functions and/or protocols that computer programmers can use when building software for a specific operating system. The API allows programmers to use predefined functions to interact with an operating system, instead of writing them from scratch. Common computer operating systems, including Windows, Unix, and the Mac OS, usually provide an API for programmers. An API is also used by hardware devices that run software programs. The API generally makes a programmer's job easier, and it also benefits the end user since it generally ensures that all programs using the same API will have a similar user interface.

The phrases “computing device” or “central processing unit” as is used herein means a computer hardware component that executes individual commands of a computer software program. It reads program instructions from a main or secondary memory, and then executes the instructions one at a time until the program ends. During execution, the program may display information to an output device such as a monitor.

The term “execute” as is used herein in connection with a computer, console, server system or the like means to run, use, operate or carry out an instruction, code, software, program and/or the like.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

It will be appreciated by persons skilled in the art that the present embodiment is not limited to what has been particularly shown and described hereinabove. A variety of modifications and variations are possible considering the above teachings without departing from the following claims.

I/We claim:

1. A computer-implemented method for assisting a consumer with healthcare enrollment, comprising:
communicating, via a computing device, a first interview prompt to a user device;
receiving, as input to the user device, a first response to the first interview prompt;
dynamically generating, via an AI-assisted reasoning engine fine-tuned on healthcare-specific data, a second interview prompt based on semantic analysis of the first response, wherein the AI-assisted reasoning engine detects incomplete or inconsistent data and generates targeted follow-up prompts;
guiding the user through the healthcare marketplace, answering enrollment-related questions, and assisting in selecting a healthcare policy based on the first response and the second response;
generating a finalized healthcare policy selection report; and
securely transmitting the finalized healthcare policy selection report to an authorized party.

2. The method of claim 1, further comprising securely transmitting the healthcare policy selection report and user data via an encrypted communication channel to an insurance provider or authorized entity, using encryption standards to ensure compliance with applicable data protection regulations.

3. The method of claim 1, wherein the AI-assisted reasoning engine is a large language model (LLM) trained to interpret natural language input, understand user needs, and generate contextually relevant follow-up prompts to optimize user engagement.

4. The method of claim 1, wherein the AI-assisted reasoning engine utilizes constitutional artificial intelligence principles configured to mitigate bias and prevent harm to users, ensuring equitable treatment across diverse demographic groups.

5. The method of claim 1, wherein the AI-assisted reasoning engine is configured to communicate with the user in one or more languages, adapting its prompts and responses to the user's preferred language.

6. The method of claim 1, wherein the AI-assisted reasoning engine provides real-time assistance, helping users understand healthcare terms, policies, and eligibility criteria by generating simplified, user-friendly responses to complex insurance-related questions.

7. The method of claim 1, further comprising generating a time-sensitive, encrypted hyperlink to the healthcare enrollment platform that requires multi-factor authentication for access and expires upon completion of the enrollment process.

8. The method of claim 7, further comprising expiring, via the computing device, the hyperlink after the consumer completes the enrollment process or after a predefined time period.

9. A computer-implemented method for simplifying the U.S. healthcare enrollment process, comprising:
communicating, via a computing device, a first healthcare-related interview prompt to a user device;
receiving, as input to the user device, a first response to the first interview prompt;
generating, via an AI-assisted reasoning engine, a report generation prompt based on the first interview prompt and the first response;
receiving, as input to the user device, a second response to the report generation prompt; and
generating, via the AI-assisted reasoning engine, an analysis and enrollment report based on the first and second responses.

10. The method of claim 9, wherein the first healthcare-related interview prompt and the report generation prompt comprise questions related to the consumer's healthcare eligibility and coverage options.

11. The method of claim 9, further comprising communicating the healthcare enrollment report to an insurance provider or healthcare agency for processing.

12. The method of claim 9, wherein generating the first healthcare-related interview prompt comprises generating the first interview prompt based on a rule-based logic system with multilingual support.

13. The method of claim 9, wherein the AI-assisted reasoning engine is configured to apply constitutional artificial intelligence principles to prevent biased or harmful outcomes based on demographic or socioeconomic data.

14. The method of claim 9, wherein the AI-assisted reasoning engine is configured to understand natural language patterns, thereby generating prompts and responses that mimic human conversation.

15. The method of claim 9, further comprising generating and communicating, via the computing device, a hyperlink for access to the healthcare enrollment process, which includes at least the first interview prompt.

16. A software product comprising at least one computer-readable storage medium having application instructions stored thereon, the application instructions executable to:
generate and communicate a hyperlink corresponding to a first interview comprising at least a first healthcare-related interview prompt;
communicate the first interview prompt to a user device;
receive, as input to the user device, a first response to the first interview prompt;
generate, via a large language model, a second interview prompt based on the first response;
receive, as input to the user device, a second response to the second interview prompt;
generate, via the large language model, a finalized healthcare enrollment report based on the first and second responses; and
expire the hyperlink after generating the healthcare enrollment report.

17. The software product of claim 16, wherein the first interview prompt and the second interview prompt comprise healthcare eligibility questions.

18. The software product of claim 16, wherein the large language model is configured to apply constitutional artificial intelligence principles to avoid bias and ensure fair treatment of users.

19. The software product of claim 16, wherein the large language model is configured to generate additional prompts that mimic human expression and adapt to the user's language or communication preferences.

20. The software product of claim 16, further comprising generating a secure, encrypted communication channel for transmitting the finalized healthcare enrollment report to an insurance provider or authorized entity, ensuring compliance with data protection and privacy regulations.
